(19) United States
(12) Patent Application Publication
(10) Pub. No.: US 2019/0017957 A1
(43) Pub. Date: Jan. 17, 2019

(54) BIOSENSOR

(71) Applicant: PHC HOLDINGS CORPORATION, Tokyo (JP)

(72) Inventors: Nobuyuki SHIGETOU, Ehime (JP); Keigo HANEDA, Ehime (JP); Shingo OOTANI, Ehime (JP)

(21) Appl. No.: 16/111,882

(22) Filed: Aug. 24, 2018

Related U.S. Application Data
(63) Continuation of application No. PCT/JP2016/077154, filed on Sep. 14, 2016.

(30) Foreign Application Priority Data
Feb. 25, 2016 (JP) 2016-034671

(51) Int. Cl. G01N 27/327; G01N 33/72; G01N 27/49

(52) U.S. Cl. CPC G01N 27/3272; G01N 27/49; G01N 33/726

(57) ABSTRACT

A biosensor includes a base substrate; an electrode layer disposed on a main surface of the base substrate and including first and second electrode pairs, each pair including a working electrode and a counter electrode; a spacer substrate on the electrode layer and having first and openings, the first opening exposing a part of the first electrode pair and the second opening exposing a part of the second electrode pair; a first reagent layer containing a first reagent reactive with a sample and disposed in the first opening; a second reagent layer containing a second reagent reactive with the sample and disposed in the second opening; and a top cover substrate on the spacer substrate, the top cover substrate including an introduction portion having at least one introduction opening, the introduction portion overlapping a part of the first opening in the spacer substrate and a part of the second opening.

This is a continuation of International Application No. PCT/JP2016/077154, with an international filing date of Sep. 14, 2016, which claims priority of Japanese Patent Application No. 2016-034671, filed on Feb. 25, 2016, the contents of each of which are incorporated herein by reference.

### BACKGROUND

This application relates to a biosensor for measuring concentration of a specific substance contained in a sample.

Biosensors have been put into practice in various fields including medical and clinical inspection and pharmaceutical manufacturing. For example, a biosensor for measuring concentration of various substances in the blood has been put into practice and is now widely used in the field of medical and clinical inspection.

Japanese Laid-Open Patent Publication No. 2015-108622 (hereinafter, referred to as “Patent Document 1”) discloses a microfluidic sensor usable as an electrochemical blood test strip for blood coagulation measurement or the like. The microfluidic sensor includes a flow path including branches in which fluids to be measured flow by the capillary action and an electrode pair provided in a reaction zone located at ends of the branches of the flow path. Patent Document 1 discloses that coagulation time is important for blood coagulation measurement and that it is important to minimize the factor preventing the flow of the fluids in the reaction zone in order to perform the measurement with high precision and high reproducibility.

### SUMMARY

It is preferable that the structure of a biosensor is optimized for the properties of a subject to be detected and the measurement method used for the detection. This disclosure provides a biosensor using an electrochemical measurement method for measuring two or more types of substances more precisely.

A biosensor according to embodiments of this disclosure includes a base substrate having a main surface; an electrode layer disposed on the main surface of the base substrate and including a first electrode pair and a second electrode pair, each pair including a working electrode and a counter electrode; a spacer substrate disposed on the electrode layer and having a first opening and a second opening formed therein, the first opening exposing a part of the first electrode pair and the second opening exposing a part of the second electrode pair, the second opening being independent from the first opening; a first reagent layer containing a first reagent reactive with a sample, the first reagent layer being disposed in the first opening; a second reagent layer containing a second reagent reactive with the sample, the second reagent layer being disposed in the second opening; and a top cover substrate disposed on the spacer substrate, the top cover substrate including an introduction portion having at least one introduction opening formed therein, the introduction portion overlapping a part of the first opening and a part of the second opening in the spacer substrate.

According to the biosensor of this disclosure, the introduction opening in the top cover substrate overlaps the first opening and the second opening. Therefore, the sample is introduced into the first opening and the second opening in which the electrodes are provided, with no need to provide a flow path including branches for introducing the sample into reaction chambers. With such a structure, the area size of the first opening and/or the second opening may be enlarged to increase the amount of the sample used for the measurement, and also the length of a border between the working electrode and the counter electrodes can be extended. Thus, the detection sensitivity of the biosensor is improved. Therefore, two or more types of substances may be measured with high precision by an electrochemical measurement method.

These general and specific aspects may be implemented using a system, a method, and a computer program, and any combination of systems, methods, and computer programs.

Additional benefits and advantages of the disclosed embodiments will be apparent from the specification and Figures. The benefits and/or advantages maybe individually provided by the various embodiments and features of the specification and drawings disclosure, and need not all be provided in order to obtain one or more of the same.

### BRIEF DESCRIPTION OF THE DRAWINGS

FIG. 1A is an isometric view of a biosensor in embodiment 1.

FIG. 1B is an exploded isometric view of the biosensor in embodiment 1.

FIG. 2A is a cross-sectional view and a plan view of the biosensor shown in FIG. 1;

FIG. 2B is a plan view of the biosensor shown in FIG. 1.

FIG. 2C is a plan view of the biosensor in the state where a top cover substrate is removed.

FIG. 2D is a plan view of the biosensor in the state where the top cover substrate and first and second reagent layers are removed.

FIG. 2E is a plan view of the biosensor in the state where a spacer substrate and components above the spacer substrate are removed.

FIG. 3 is a block diagram showing a structure of a measurement device.

FIG. 4 is an isometric view showing how a sample is dropped to the biosensor.

FIG. 5 is a graph showing the relationship between the concentration of HbAlc and the current value in the case where measurement is performed on HbAlc, the concentration of which is known, by use of the biosensor in embodiment 1.

FIG. 6 is a graph showing the relationship between the concentration of Hb and the current value in the case where measurement is performed on Hb, the concentration of which is known, by use of the biosensor in embodiment 1.

FIG. 7A is an isometric view of a biosensor in embodiment 2.

FIG. 7B is an exploded isometric view of the biosensor in embodiment 2.

FIG. 8 is an exploded isometric view of a biosensor in embodiment 3.

FIG. 9 is an exploded isometric view of a biosensor in embodiment 4.

### DETAILED DESCRIPTION

The present inventors studied, in detail, the structure of a biosensor capable of measuring two or more types of substances by an electrochemical measurement method with high precision. It is now assumed that, for example, the value of hemoglobin Alc (hereinafter, referred to as “HbAlc”) is to be measured electrochemically by a biosensor. The HbAlc value is an inspection value that shows the ratio of hemoglobin bonded with sugar (HbAlc) with respect to hemoglobin in the erythrocytes (referred to as “Hb”). The HbAlc value reflects an average blood sugar level in the past one to two months, and therefore, is less likely to be influenced by the pre-inspection meal than the blood glucose level and thus is usable as a more accurate index for diabetes control. In order to find the HbAlc value, the concentration (amount) of HbAlc and the concentration (amount) of Hb need to be found. The term “Hb” encompasses HbAlc and all the other derivatives. The concentration of HbAlc is relatively low and thus is difficult to be measured especially electrochemically. Therefore, the HbAlc value has been measured mainly by an optical spectroscopic method.

In the case where the concentration of the substance to be measured by a biosensor using an electrochemical method is low, it is conceivable to use a maximum possible amount of sample and to enlarge a detection region of the electrode to increase the detection precision. A conventional biosensor for detecting two or more types of substances divides the sample into two or more by using a microfluidic path including branches and detects the substances in the sample in each of reaction chambers by using an electrochemical reaction. However, the biosensor of such a structure, in which a microfluidic path including branches is formed, may occasionally not allow formation of a large electrode. In light of such a problem, the present inventors conceived a biosensor having a novel structure. The overview of the biosensor in this disclosure is as follows.

[Item 1]

A biosensor includes a base substrate, an electrode layer, a spacer substrate, a first reagent layer, a second reagent layer and a top cover substrate. The base substrate has a main surface. The electrode layer is disposed on the main surface of the base substrate and includes a first electrode pair and a second electrode pair, each pair including a working electrode and a counter electrode. The spacer substrate is disposed on the electrode layer and has a first opening and a second opening formed therein. The first opening exposes a part of the first electrode pair and the second opening exposes a part of the second electrode pair. The second opening is independent from the first opening. The first reagent layer contains a first reagent reactive with a sample, and the first reagent layer is disposed in the first opening. The second reagent layer contains a second reagent reactive with the sample, and the second reagent layer is disposed in the second opening. The top cover substrate is disposed on the spacer substrate. The top cover substrate includes an introduction portion having at least one introduction opening formed therein. The introduction portion overlaps a part of the first opening in the spacer substrate and a part of the second opening in the spacer substrate.

According to this structure, the introduction opening in the top cover substrate overlaps the first opening and the second opening. Therefore, the sample is introduced into the first opening and the second opening in which the electrodes are provided, with no need to provide a flow path including branches for introducing the sample into reaction chambers. With such a structure, the area size of the first opening and/or the second opening may be enlarged to increase the amount of the sample used for the measurement, and also the length of a border between the working electrode and the counter electrodes can be extended. Thus, the detection sensitivity of the biosensor is improved. Since the sample is introduced from the introduction portion, the sample is introduced into the first opening and the second opening provided in the spacer substrate at the same time in parallel to each other. Therefore, the time required to fill the first opening and the second opening with the sample to start the measurement is shortened. Since the first opening and the second opening are independent from each other, the size of each thereof may be set arbitrarily. The amounts of the sample to be used for the first opening and the second opening are adjusted independently. Therefore, such amounts of the sample may be made different in accordance with the concentrations, to be detected, of the substances in the sample, and also in accordance with the detection sensitivity on each of the substances. The measurement is performed with appropriate sensitivity.

The top cover substrate includes the introduction portion. Therefore, the amount of the sample to be dropped may be increased to fill the first opening and the second opening with the sample within a short time. This shortens the measurement time. Even if the area size of each of the first electrode pair and the second electrode pair for the measurement is enlarged, the sample of a sufficient amount is put into contact with the electrode pairs within a short time. This improves the detection sensitivity.

[Item 2]

The biosensor according to item 1, wherein in the spacer substrate, the first opening has an area size larger than an area size of the second opening.

With this structure, the first opening is large. Therefore, a larger amount of sample may be used for the measurement, and thus the detection precision of the substance to be detected by use of the first substance is improved.

[Item 3]

The biosensor according to item 1 or 2, wherein in the electrode layer, a length of a border between the working electrode and the counter electrode adjacent thereto of the first electrode pair exposed to the first opening is longer than a length of a border between the working electrode and the counter electrode adjacent thereto of the second electrode pair exposed to the second opening.

With this structure, the length of the border between the working electrode and the counter electrode of the first electrode pair may be extended. This improves the detection sensitivity.

[Item 4]

The biosensor according to item 1, further comprising a first discharge opening and a second discharge opening respectively in communication with the first opening and the second opening in the spacer substrate.

With this structure, the first discharge opening and the second discharge opening are provided. This allows the sample to be introduced into the first opening and the second opening with certainty by use of the capillary force.

[Item 5]

The biosensor according to item 4, wherein the first discharge opening and the second discharge opening are provided in the spacer substrate, each have an opening at a side surface of the spacer substrate, and are respectively connected with the first opening and the second opening.

With this structure, the top cover substrate merely has the introduction opening formed in a main surface thereof. Therefore, it is less possible that any foreign object enters the first discharge opening or the second discharge opening or that the operator considers, by mistake, the first discharge opening or the second discharge opening as an introduction opening and introduces the sample through the first discharge opening or the second discharge opening.

[Item 6]

The biosensor according to item 4, wherein the first discharge opening and the second discharge opening are provided in the top cover substrate and respectively overlap another part of the first opening in the spacer substrate and another part of the second opening in the spacer substrate.

[Item 7]

The biosensor according to any one of items 1 through 6, wherein the introduction portion of the top cover substrate has one introduction opening formed therein, and the introduction opening overlaps the part of the first opening in the spacer substrate and the part of the second opening in the spacer substrate.

With this structure, the sample may be introduced from the introduction opening into the first opening and the second opening at the same time. This simplifies the operation for the measurement.

[Item 8]

The biosensor according to any one of items 1 through 6, wherein the introduction portion of the top cover substrate has two introduction openings formed therein, one of the introduction openings overlaps the part of the first opening in the spacer substrate, and the other of the introduction openings overlaps the part of the second opening in the spacer substrate.

With this structure, the introduction openings are respectively provided for the first opening and the second opening in the spacer substrate. Therefore, the capillary forces used to introduce the sample into the first opening and the second opening are more easily adjustable independently. Even in the case where the amounts of the sample to be introduced into the first opening and the second opening are increased because of the difference in the detection sensitivity, the time required for the introduction may be made the same, and the sample is allowed to be introduced into the entirety of the first opening and the second opening with certainty. This shortens the measurement time and realizes the measurement with high reproducibility.

[Item 9]

The biosensor according to item 1, wherein:
the base substrate, the spacer substrate and the top cover substrate each have a strip shape that is longer in a first direction than in a second direction perpendicular to the first direction;
the first opening and the second opening in the spacer substrate are disposed adjacent to each other in the first direction; and
the introduction portion of the top cover substrate has one introduction opening formed therein, and the introduction opening overlaps parts of the first opening and the second opening that are adjacent to each other.

[Item 10]

The biosensor according to item 9, wherein:
the top cover substrate further has a first discharge opening and a second discharge opening formed therein;
the first discharge opening and the introduction opening in the introduction portion respectively overlap two ends in the first direction of the first opening in the spacer substrate; and
the second discharge opening and the introduction opening in the introduction portion respectively overlap two ends in the first direction of the second opening in the spacer substrate.

[Item 11]

The biosensor according to any one of items 1 through 10, wherein:
the sample contains hemoglobin separated from erythrocytes;
the hemoglobin contains hemoglobin Alc;
the first reagent is specifically reactive with the hemoglobin Alc or a substance derived from the hemoglobin Alc; and
the second reagent is reactive with the hemoglobin.

Hereinafter, embodiments of a biosensor in this disclosure will be described in detail with reference to the drawings. The biosensor of this disclosure electrochemically detects and/or quantifies two or more different substances contained in a sample. In each of the embodiments of this disclosure, one embodiment of a biosensor detecting a first substance and a second substance contained in the sample will be described. Especially in the case where the detection sensitivities on two or more substances contained in the sample are different, a substance with relatively low detection sensitivity may be detected with improved sensitivity by the biosensor of this disclosure. In the following embodiments, the first substance and the second substance are respectively HbAlc and Hb obtained by a preprocess performed on the blood, and the biosensor electrochemically measures the concentrations of the first substance and the second substance. The biosensor of this disclosure is preferably usable to detect any other appropriate substance. The following embodiments are merely illustrative, and the present invention is not limited to any of the following embodiments. In the following embodiments, components are represented by reference signs in the drawings, and substantially the same descriptions may be omitted or components that are not referred to in the description may not be represented by any reference sign, for easier understanding and or avoidance of unnecessary repetitions.

### Embodiment 1

<Structure of the Biosensor>

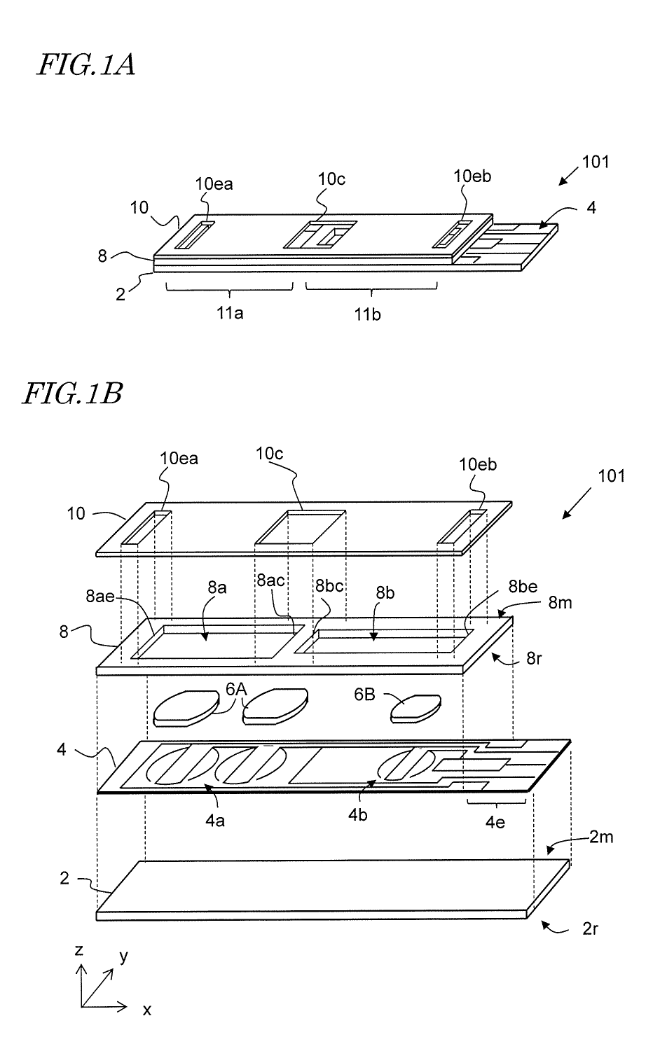

A biosensor in embodiment 1 in this disclosure will be described. FIG. 1A and FIG. 1B are respectively an isometric view and an exploded isometric view of a biosensor 101 in this embodiment. The biosensor 101 includes a base substrate 2, an electrode layer 4, first and second reagent layers 6A and 6B, a spacer substrate 8, and a top cover substrate 10. As shown in FIG. 1B, the base substrate 2, the spacer substrate 8 and the top cover substrate 10 each have a strip shape and are each longer in a longitudinal direction thereof, which is along the x direction, than in a width direction, which is along the y direction.

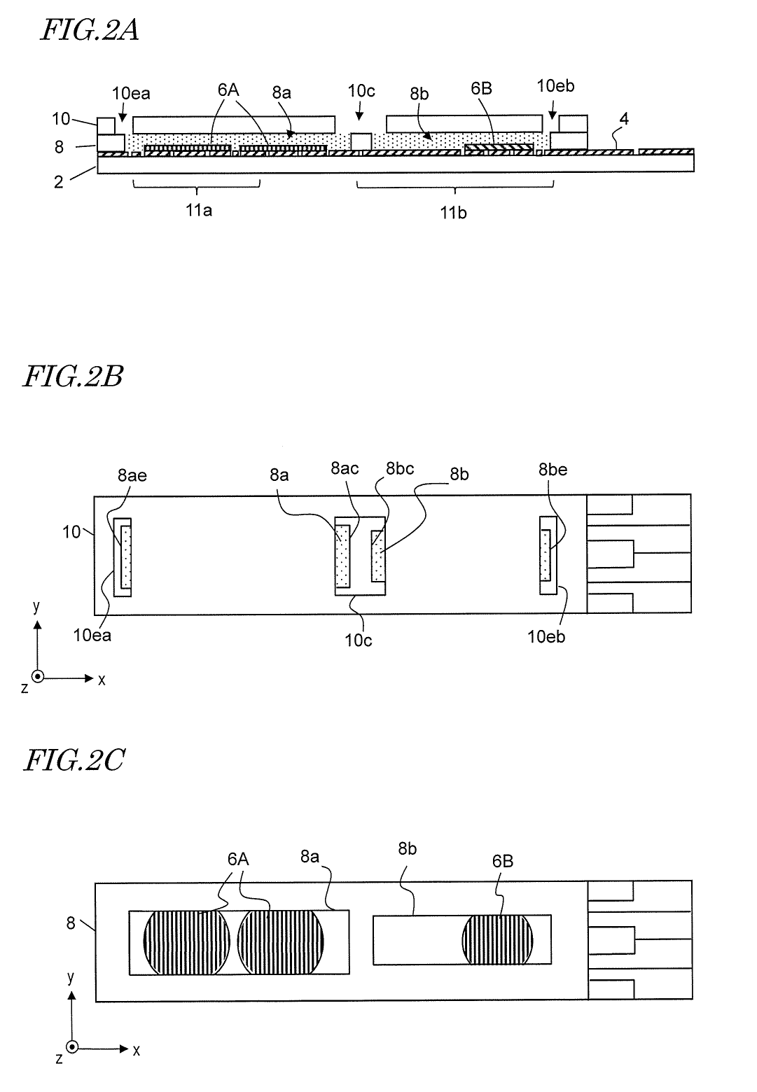

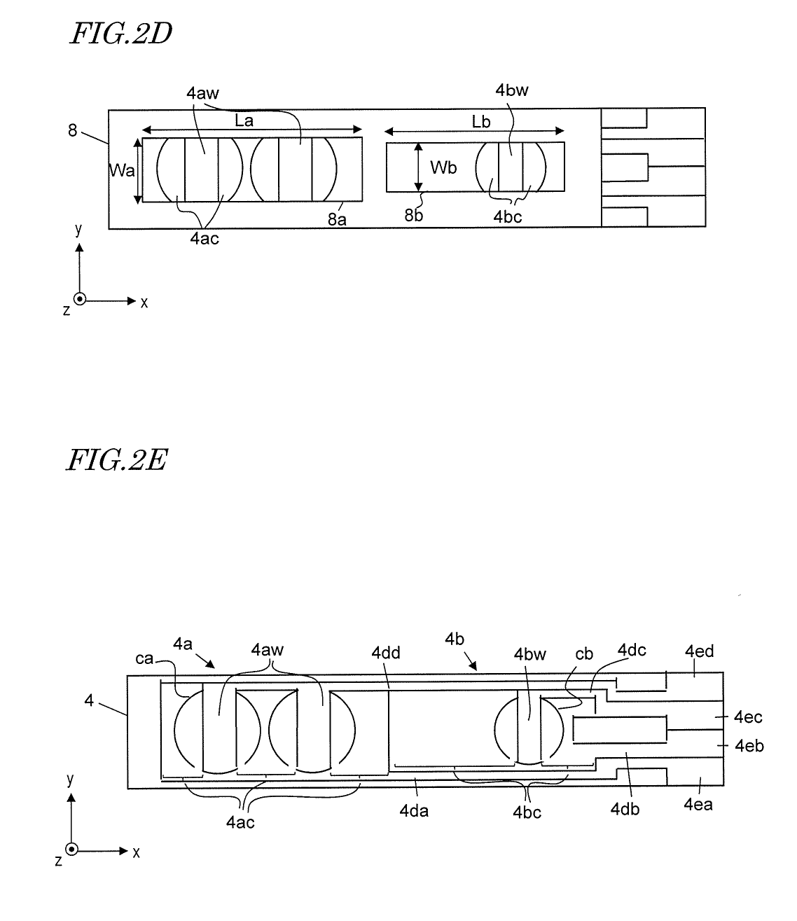

FIG. 2A and FIG. 2B are respectively a cross-sectional view and a plan view of the biosensor 101. FIG. 2C, FIG. 2D and FIG. 2E are respectively a plan view of the biosensor 101 in the state where the top cover substrate 10 is removed, a plan view of the biosensor 101 in the state where the top cover substrate 10 and the first and second reagent layers 6A and 6B are removed, and a plan view of the biosensor 101 in the state where the spacer substrate 8 and components above the spacer substrate 8 are removed. With reference to these figures, a structure of the biosensor 101 will be described in detail.

The base substrate 2 supports all the other components of the biosensor 101. The base substrate 2 has a first main surface 2m and a second main surface 2r, and has a strip shape as described above. A “main surface” of a substrate is a main large surface by which the substrate supports components.

The electrode layer 4 is disposed on the first main surface 2m of the base substrate 2. The electrode layer 4 includes at least a first electrode pair 4a and a second electrode pair 4b. The first electrode pair 4a and the second electrode pair 4b are used to detect a first substance and a second substance, which are different substances contained in a sample. In this embodiment, the first substance is HbAlc, and the second substance is Hb.

As shown in FIG. 2E, the first electrode pair 4a includes working electrodes 4aw and counter electrodes 4ac. In this embodiment, the first electrode pair 4a includes two working electrodes 4aw and three counter electrodes 4ac. The two working electrodes 4aw are connected to a terminal 4ed via a line 4dd. The three counter electrodes 4ac are connected to a terminal 4ea via a line 4da. In the x direction, which is the longitudinal direction of the base substrate 2, the working electrodes 4aw and the counter electrodes 4ac are disposed alternately.

Similarly, the second electrode pair 4b includes a working electrode 4bw and counter electrodes 4bc. In this embodiment, the second electrode pair 4b includes one working electrode 4bw and two counter electrodes 4bc. The working electrode 4bw is connected to a terminal 4ec via a line 4dc. The two counter electrodes 4bc are connected to a terminal 4eb via a line 4db. In the longitudinal direction of the base substrate 2, the working electrode 4bw and the counter electrodes 4bc are disposed alternately. A pair of counter electrodes 4ac having each of the working electrodes 4aw therebetween each have an arcuate cut ca. Similarly, the pair of counter electrodes 4bc having the working electrode 4bw therebetween each have an arcuate cut cb.

In this embodiment, the above-described structure of the electrodes in the electrode layer 4 is provided by patterning a metal film covering substantially the entirety of the main surface 2m of the base substrate 2.

The spacer substrate 8 is disposed on the electrode layer 4, and has a first opening 8a and a second opening 8b formed therein. The spacer substrate 8 is shorter than the base substrate 2 in the longitudinal direction. Therefore, the terminals 4ea through 4ed are not covered by the spacer substrate 8 and are exposed.

The first opening 8a and the second opening 8b are through-holes reaching two main surfaces 8m and 8r of the spacer substrate 8. The first opening 8a and the second opening 8b are independent spaces from each other and are not in communication with each other. The spacer substrate 8 is provided between the base substrate 2 and the top cover substrate 10, and thus the first opening 8a and the second opening 8b act as reaction chamber holding a sample and detecting an electric change of the sample caused by reactions of the sample with the first reaction layers 6A and 6B.

The first opening 8a and the second opening 8b each have a rectangular shape having a longitudinal direction in the longitudinal direction of the spacer substrate 8, and are arranged in the longitudinal direction of the spacer substrate 8. Therefore, one end 8ac of the first opening 8a is close to one end 8bc of the second opening 8b at the center or in the vicinity thereof of the spacer substrate 8. The other end 8ae of the first opening 8a and the other end 8be of the second opening 8b are respectively close to two ends, in the longitudinal direction, of the spacer substrate 8.

In this embodiment, the first substance in the sample held in the first opening 8a is detected with high sensitivity. Namely, the reaction chamber provided to detect the first substance has a larger capacity than the capacity of the reaction chamber provided to detect the second substance. Therefore, it is preferable that the first opening 8a has an area size larger than the area size of the second opening 8b. In this embodiment, as shown in FIG. 2D, where the length of the first opening 8a in the x direction (longitudinal direction) is La, the length of the second opening 8b in the x direction (longitudinal direction) is Lb, the width of the first opening 8a in the y direction (direction perpendicular to the longitudinal direction) is Wa, and the width of the second opening 8b in the y direction (direction perpendicular to the longitudinal direction) is Wb, La>Lb and Wa>Wb. With such an arrangement, the amount of the sample that can be held in the first opening 8a may be made larger than the amount of the sample that can be held in the second opening 8b.

The sizes of the first opening 8a and the second opening 8b may be determined in accordance with the concentrations of the first substance and the second substance in the sample and the required amount of the sample determined in consideration of, for example, the detection sensitivity by the detection method to be used. In the case where the sample is introduced into the first opening 8a and the second opening 8b by use of the capillary force (capillary action), the spacer substrate 8 has a thickness (z direction) with which the capillary force may act on the sample.

Since the spacer substrate 8 is disposed on the electrode layer 4, a part of the first electrode pair 4a and a part of the second electrode pair 4b are respectively exposed to the first opening 8a and the second opening 8b. Electric or electrochemical changes of the sample held in the first opening 8a and the second opening 8b may be respectively detected by the first electrode pair 4a and the second electrode pair 4b. In the first opening 8a, a total length of borders between the working electrodes 4aw and the counter electrodes 4ac of the first electrode pair 4a is 4×Wa. In the second opening 8b, a total length of borders between the working electrode 4bw and the counter electrodes 4bc of the first electrode pair 4b is 2×Wb. Namely, the total length of the borders between the working electrode (s) and the counter electrodes is longer in the first electrode pair 4a than in the second electrode pair 4b . The first electrode pair 4a has higher detection sensitivity.

The first reagent layer 6A and the second reagent layer 6B respectively contain a first reagent and a second reagent. The first reagent and the second reagent react with the first substance and the second substance in the sample, respectively, or with substances derived from the first substance and the second substance by preprocessing, to cause an electrically detectable change to the sample. In this embodiment, the first reagent is fructosyl peptide oxidase, which is a detection enzyme specifically reactive with fructosyl valyl histidine, which is generated by a preprocess performed on HbAlc, and is immobilized to the first reagent layer. The second reagent is potassium ferricyanide, which is a mediator reactive with any type of Hb encompassing HbAlc, and is immobilized to the second reagent layer.

The first reagent layer 6A and the second reagent layer 6B are respectively disposed in the first opening 8a and the second opening 8b in the spacer substrate 8. More specifically, the first reagent layer 6A is disposed above an area in the vicinity of the borders between the working electrodes 4aw and the counter electrodes 4ac of the two first electrode pairs 4a, and the second reagent layer 6B is disposed above an area in the vicinity of the borders between the working electrode 4bw and the counter electrodes 4bc of the second electrode pair 4b. In this embodiment, in the case where the materials of the first reagent layer 6A and the second reagent layer 6B are dropped from above the electrode layer 4, regions in which the materials expand are defined by the arcked cuts ca and cb formed in the counter electrodes. Therefore, the amount of the first reagent layer 6A disposed in the vicinity of the borders between the working electrodes and the counter electrodes, and the amount of the second reagent layer 6B disposed in the vicinity of the borders between the working electrode and the counter electrodes, are made the same as each other. As a result, the level of the electric current is suppressed from being varied between biosensor cells at the time of detection of the first substance and the second substance.

The top cover substrate 10 is disposed on the spacer substrate 8. The top cover substrate 10 includes an introduction portion having at least one introduction opening formed therein. In this embodiment, the introduction portion has one introduction opening 10c formed therein. As well shown in FIG. 2A and FIG. 2B, the introduction opening 10c overlaps a part of the first opening 8a and a part of the second opening 8b. More specifically, the one end 8ac, in the longitudinal direction, of the first opening 8a and the one end 8bc, in the longitudinal direction, of the second opening 8b are disposed in the introduction opening 10c. Therefore, the introduction opening 10c is in communication with the first opening 8a and the second opening 8b. When the sample is dropped to the introduction opening 10c, the sample flows into the first opening 8a and the second opening 8b.

In this embodiment, the top cover substrate 10 further has a first discharge opening 10ea and a second discharge opening 10eb formed therein. The first discharge opening 10ea and the second discharge opening 10eb respectively overlap a part of the first opening 8a and a part of the second opening 8b. The other end 8ae of the first opening 8a and the other end 8be of the second opening 8b are respectively disposed in the first discharge opening 10ea and the second discharge opening 10eb. Since the first discharge opening 10ea and the second discharge opening 10eb are provided, the following occurs. In the case where the biosensor 101 is configured such that the capillary force acts on the first opening 8a and the second opening 8b, gas present in the first opening 8a and the second opening 8b is discharged from the first discharge opening 10ea and the second discharge opening 10eb as the sample is pulled into the first opening 8a and the second opening 8b. In this manner, the sample is introduced into the first opening 8a and the second opening 8b by the capillary force. In the case where the biosensor 101 is configured such that the capillary force does not substantially act on the first opening 8a or the second opening 8b, neither the first discharge opening 10ea nor the second discharge opening 10eb needs to be provided. In this case, the sample introduced from the introduction opening 10c flows into the first opening 8a and the second opening 8b by the gravity.

The above-described components are stacked, and as a result, as shown in FIG. 1A and FIG. 2A, a first cell 11a having the introduction opening 10c, the first opening 8a and the first discharge opening 10ea faulted therein and detecting the first substance, and a second cell 11b having the introduction opening 10c, the second opening 8b and the second discharge opening 10eb faulted therein and detecting the second substance, are provided.

The base substrate 2, the spacer substrate 8 and the top cover substrate 10 are each formed of an insulating material. As the insulating material, any of various resin materials, for example, PET (poly (ethylene terephthalate)) or the like is usable. Instead of such a resin material, an inorganic material such as glass or the like is usable. The electrode layer 4 is formed of a conductive material. The electrode layer 4 is formed by patterning a metal film of, for example, palladium or the like with a laser beam or the like. The base substrate 2, the electrode layer 4, the spacer substrate 8 and the top cover substrate 10 are bonded together with, for example, an adhesive.

<Structure of the Measurement Device>

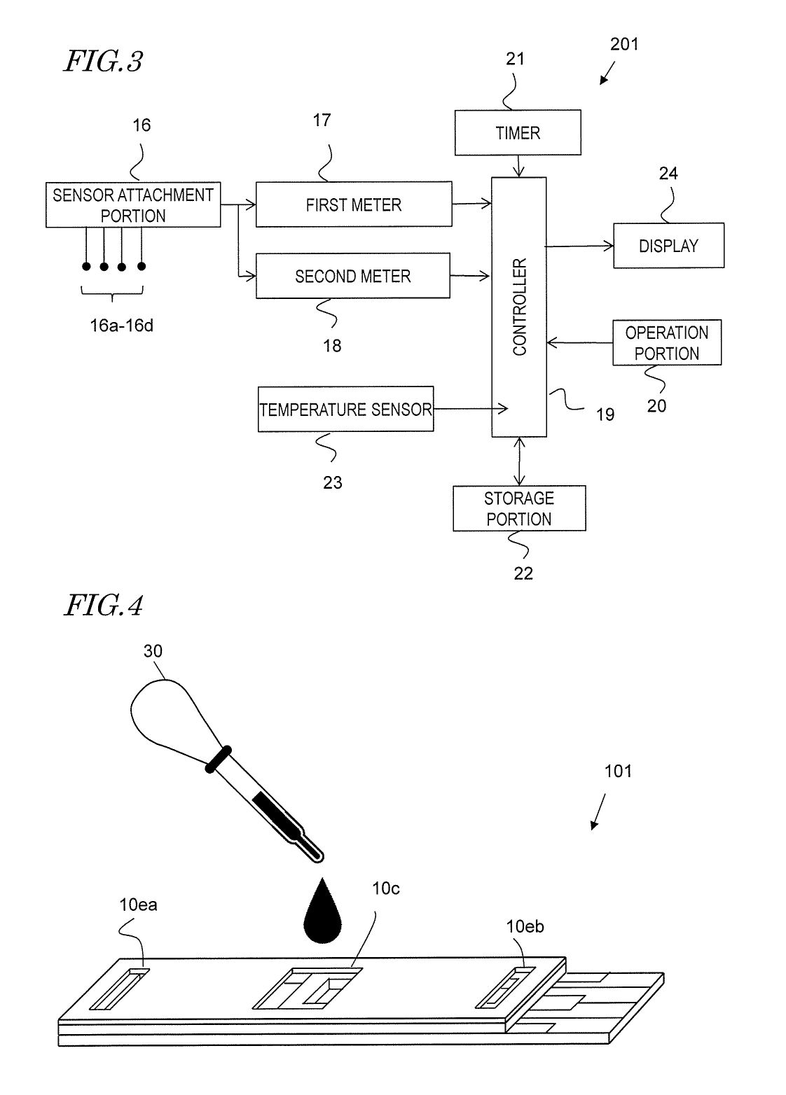

FIG. 3 shows an example of structure of a measurement device 201 for measuring the first substance and the second substance contained in the sample by having the biosensor 101 mounted thereon. The measurement device 201 includes a sensor attachment portion 16, a first meter 17, a second meter 18, and a controller 19. The sensor attachment portion 16 includes connectors 16a through 16d in contact with the terminal 4ea through 4ed of the biosensor 101 and electrically connected with the first meter 17 and the second meter 18. The first meter 17 and the second meter 18 apply a predetermined voltage to the first electrode pair 4a and the second electrode pair 4b via the connectors 16a through 16d and the terminals 4ea through 4ed to measure the current values. The measured current values are output to the controller 19.

The controller 19 determines the concentrations of the first substance and the second substance from the current values (or voltage values) received from the first meter 17 and the second meter 18. For example, the controller 19 has, stored thereon, a table showing the relationship between the current values and the concentration values or a function of the current values and the concentration values, and calculates the concentrations of the first substance and the second substance based on the table or the function. In this embodiment, the first substance is HbAlc and the second substance is Hb. The ratio of HbAlc with respect to Hb is calculated based on the current values or the concentration values, and the HbAlc value is determined. The measurement device 201 also includes an operation portion 20, a timer 21, a storage portion 22, a temperature sensor 23 and a display 24. The measurement device 201 may refer to information from the temperature sensor 23 to correct the HbAlc value. The corrected HbAlc value is stored on the storage portion 22 and displayed on the display 24, together with information on the time supplied by the timer 21. The controller 19 performs processes such as the measurement, display, storage of the measurement results and the like based on an instruction input by an operator to the operation portion 20.

The controller 19 of the measurement device 201 includes a computation device such as a microcomputer or the like and a storage portion, such as a memory. The storage portion stores a program defining a procedure of the measurement. The computation device executes the program, and thus the concentrations of the first substance and the second substance are measured. The first meter 17 and the second meter 18 each include a power supply circuit generating a predetermined voltage and a current meter measuring a current value. A measured current value is, for example, converted into a digital signal and processed as described above by the controller 19. The display 24 may be a liquid crystal display device, an organic EL display device or the like.

<Measurement of HbAlc by the Biosensor 101 and the Measurement Device 201>

Hereinafter, a specific procedure of measurement performed on HbAlc by use of the biosensor 101 and the measurement device 201 will be described.

(1) Preprocess on the Sample

First, a blood sample obtained from a measurement subject person is preprocessed. Specifically, a hemolytic agent such as a surfactant or the like is added to the obtained blood sample to elute Hb in the erythrocytes in the blood sample.

Next, protease, which is a degradative enzyme, is added to the blood sample containing the eluted Hb to degrade lib. As a result of the degradation, HbAlc in Hb generates fructosyl valyl histidine. Hereinafter, the preprocessed blood sample will be referred to simply as a “sample”.

(2) Electrochemical Reaction Caused by the Biosensor 101

The biosensor 101 is attached to the measurement device 201. The sample is absorbed by a dropper 30, and as shown in FIG. 4, is dropped to an area in the vicinity of the introduction opening 10c of the biosensor 101. The sample attached to the area in the vicinity of the introduction opening 10c is pulled through the introduction opening 10c by the capillary force and is introduced into the first opening 8a and the second opening 8b at the same time, namely, in parallel to each other.

Fructosyl valyl histidine in the sample introduced into the first opening 8a reacts with fructosyl peptide oxidase, which is the first reagent contained in the first reagent layer 6A, to generate hydrogen peroxide. The conductivity of the sample varies in accordance with the amount of the generated hydrogen peroxide. Therefore, the value of the current flowing between the working electrodes 4aw and the counter electrodes 4ac of the first electrode pair 4a also varies in accordance with the amount of the generated hydrogen peroxide. Since fructosyl valyl histidine is derived from HbAlc, the current value varies in accordance with the amount HbAlc in the sample.

Hb in the sample introduced into the second opening 8b and potassium ferricyanide, which is the second reagent contained in the second reagent layer 6B, cause an oxidation-reduction reaction. The movement of charges caused by the oxidation-reduction reaction is detected as a current flowing between the working electrode 4bw and the counter electrodes 4bc of the second electrode pair 4b, and thus the current value in accordance with the amount of Hb is detected.

(3) Measurement by the Measurement Device 201

For example, a voltage of 0.1 V to 0.4 V is applied by the measurement device 201 for 5 to 10 seconds between the working electrodes 4aw and the counter electrodes 4ac of the first electrode pair 4a, and the amount of the current flowing between the working electrodes 4aw and the counter electrodes 4ac is measured. Similarly, a voltage of 0.1 V to 0.4 V is applied for 5 to 10 seconds between the working electrode 4bw and the counter electrodes 4bc of the second electrode pair 4b, and the amount of the current flowing between the working electrode 4bw and the counter electrodes 4bc is measured.

The current values measured by the first meter 17 and the second meter 18 respectively correspond to the amount of HbAlc and the amount of lib. The measurement device 201 uses these values to find the HbAlc value.

<Measurement Result Examples>

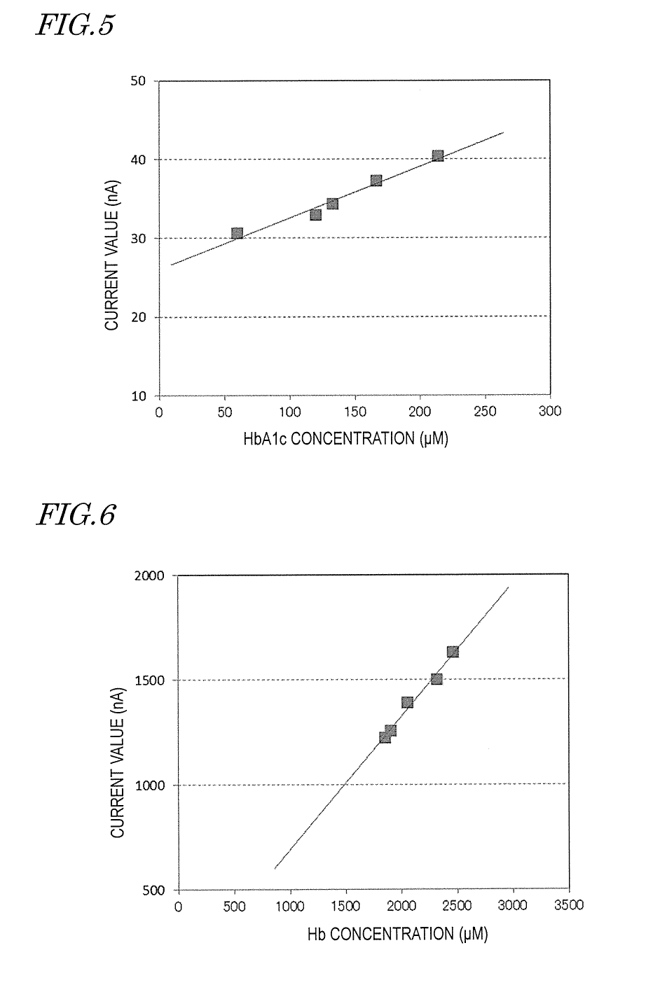

FIG. 5 and FIG. 6 show the results of the measurement performed on samples, the concentrations of HbAlc and Hb of which are known. Five types of samples, the concentrations of HbAlc of which were known in advance, were used to measure the current values by the first meter 17. In the graph of FIG. 5, the concentrations of HbAlc of the samples are represented by the horizontal axis, and the measured current values are represented by the vertical axis. Similarly, five types of samples, the concentrations of Hb of which were known in advance, were used to measure the current values by the second meter 18. In the graph of FIG. 6, the concentrations of Hb of the samples are represented by the horizontal axis, and the measured current values are represented by the vertical axis.

As seen from FIG. 5 and FIG. 6, the concentration of HbAlc and the current value have a linear relationship, and the concentration of Hb and the current value also have a linear relationship. It is seen from this that the biosensor 101 of this disclosure is usable to electrochemically measure the concentration of HbAlc and the concentration of Hb and find the HbAlc value with high precision.

<Effect, etc.>

As described above, in the biosensor of this disclosure, the introduction opening in the top cover substrate overlaps the first opening and the second opening. Therefore, the sample is introduced into the first opening and the second opening in which the electrodes are provided, with no need to provide a flow path including branches for introducing the sample into the reaction chambers. Namely, the biosensor does not include a flow path including branches. Therefore, the area size of the first opening and/or the second opening may be enlarged to increase the amount of the sample used for the measurement, and also the length of the borders between the working electrode (s) and the counter electrodes may be extended. Thus, the detection sensitivity of the biosensor is improved. Since the sample is introduced from the introduction portion, the sample is introduced into the first opening and the second opening provided in the spacer substrate at the same time in parallel to each other. Therefore, the time required to fill the first opening and the second opening with the sample to start the measurement is shortened.

The sample is reacted with the first substance and the second substance respectively in the first opening and the second opening. Therefore, the two types of substances in the sample are detected or quantified by use of the first electrode pair and the second electrode pair. Since the first opening and the second opening are independent from each other, the measurement on either one of the first substance or the second substance does not influence the measurement on the other substance. The two types of substances are detected independently with high precision.

Since the first opening and the second opening are independent from each other, the size of each thereof may be set arbitrarily. The amounts of the sample to be used for the first opening and the second opening are adjusted independently. Therefore, such amounts of the sample are made different in accordance with the concentrations, to be detected, of the substances in the sample, and also in accordance with the detection sensitivity on each of the substances. The measurement is performed with appropriate sensitivity. For example, the area size of the first opening may be made larger than that of the second opening, so that the detection precision on the substance to be detected by use of the first substance is improved. The area size of the first opening may be made larger than that of the second opening to extend the length of the borders between the working electrodes and the counter electrodes of the first electrode pair, so that the detection sensitivity thereof is further improved. The first opening and the second opening are independent from each other. Therefore, in the case where the sample is introduced into the first opening and the second opening by the capillary force, the capillary force acting on each of the first opening and the second opening is independently adjustable.

The top cover substrate includes the introduction portion. Therefore, the amount of the sample to be dropped may be increased to fill the first opening and the second opening with the sample within a short time. This shortens the measurement time. Even if the area size of each of the first electrode pair and the second electrode pair for the measurement is enlarged, the sample of a sufficient amount is put into contact with the electrode pairs within a short time. This improves the detection sensitivity.

The first discharge opening and the second discharge opening are provided respectively for the first opening and the second opening. This allows the sample to be introduced into the first opening and the second opening by use of the capillary force. Therefore, the first opening and the second opening are filled with the sample stably and certainly.

### Embodiment 2

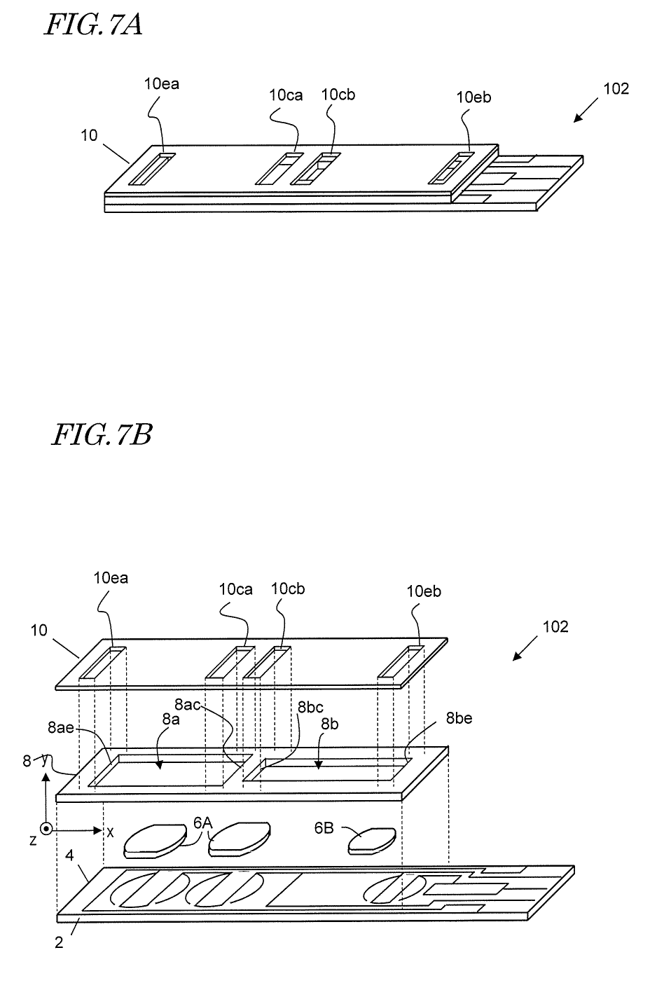

A biosensor in embodiment 2 of this disclosure will be described. FIG. 7A and FIG. 7B are respectively an isometric view and an exploded isometric view of a biosensor 102 in this embodiment. Unlike in the biosensor 101 in embodiment 1, in the biosensor 102, the top cover substrate 10 includes an introduction portion having a first introduction opening 10ca and a second introduction opening 10cb formed therein. The one end 8ac of the first opening 8a is disposed in the first introduction opening 10ca, and the one end 8bc of the second opening 8b is disposed in the second introduction opening 10cb. The first introduction opening 10ca and the second introduction opening 10cb are independent from each other and are not in communication with each other. Therefore, the area size (size) of each introduction opening is independently adjustable. The area sizes of the first introduction opening 10ca and the second introduction opening 10cb respectively overlapping the first opening 8a and the second opening 8b may be varied, so that the magnitudes of the capillary forces acting on the first opening 8a and the second opening 8b are independently adjusted. Even if the first opening 8a and the second opening 8b are different in the area size or the shape, the capillary force acting on each of the first opening 8a and the second opening 8b is adjustable to be appropriate. Thus, the sample is introduced into the biosensor 102 more certainly.

The first introduction opening 10ca and the second introduction opening 10cb are close to each other. Therefore, for example, the sample, when being dropped to an area between the first introduction opening 10ca and the second introduction opening 10cb, is introduced into both of the first introduction opening 10ca and the second introduction opening 10cb, and thus is introduced into the first opening 8a and the second opening 8b substantially at the same time.

### Embodiment 3

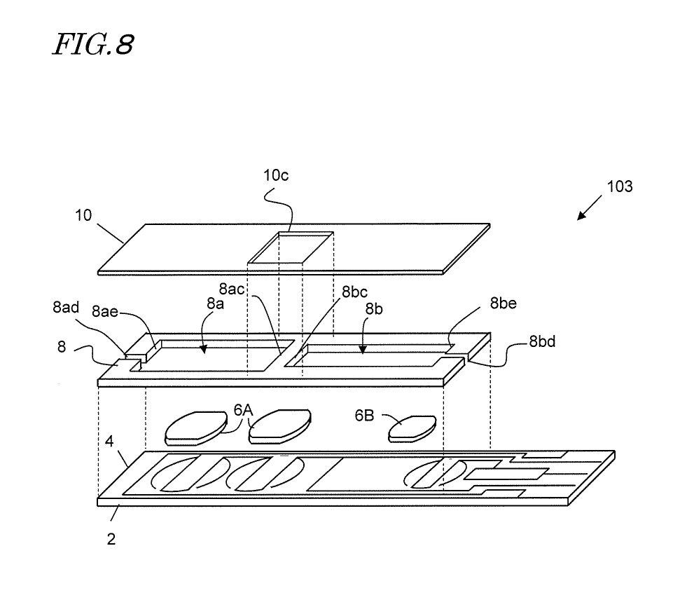

A biosensor in embodiment 3 of this disclosure will be described. FIG. 8 is an exploded isometric view of a biosensor 103 in this embodiment. Unlike in the biosensor 101 in embodiment 1, in the biosensor 103, the spacer substrate 8 has a first discharge opening 8ad and a second discharge opening 8bd formed therein. The first discharge opening 8ad is provided at the other end 8ae of the first opening 8a and has an opening at one side surface, in the longitudinal direction, of the spacer substrate 8. The second discharge opening 8bd is provided at the other end 8be of the second opening 8b and has an opening at the other side surface, in the longitudinal direction, of the spacer substrate 8.

With this structure, the top cover substrate 10 merely has the introduction opening 10c formed in a main surface thereof. Therefore, it is less possible that any foreign object enters the first discharge opening 10ea or the second discharge opening 10eb or that the operator considers, by mistake, the first discharge opening 10ea or the second discharge opening 10eb as an introduction opening and introduces the sample through the first discharge opening 10ea or the second discharge opening 10eb.

### Embodiment 4

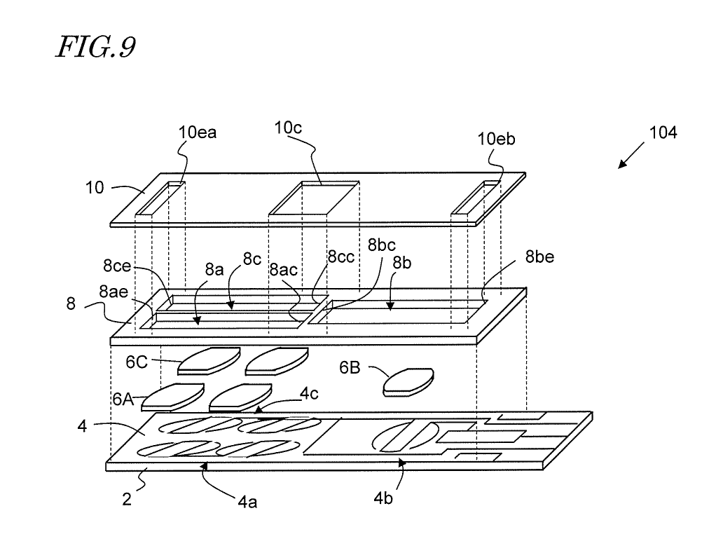

A biosensor in embodiment 4 of this disclosure will be described. FIG. 9 is an exploded isometric view of a biosensor 104 in this embodiment. Unlike in the biosensor 101 in embodiment 1, in the biosensor 104, the spacer substrate 8 has the first opening 8a, the second opening 8b and a third opening 8c faulted therein, the electrode layer 4 further includes a third electrode pair 4c, and the biosensor 104 includes a third reagent layer 6C. The first opening 8a and the third opening 8c are arranged in a direction perpendicular to the longitudinal direction. The one end 8ac of the first opening 8a and one end 8cc of the third opening 8c are close to the one end 8bc of the second opening 8b. The introduction opening 10c overlaps the one end 8ac of the first opening 8a, the one end 8cc of the third opening 8c and the one end 8bc of the second opening 8b. The first discharge opening 10ea overlaps the other end 8ae of the first opening 8a and the other end 8ce of the third opening 8c. The third electrode pair 4c is partially exposed to the third opening 8c. The third reagent layer 6C is disposed in the third opening 8c.

With the biosensor 104, the sample is introduced from the introduction portion of the top cover substrate 10 and thus is introduced into the first opening 8a, the second opening 8b and the third opening 8c provided in the spacer substrate 8 in parallel to each other. The third reagent layer 6C may contain a substance reactive with a third substance contained in the sample or a substance derived from the third substance, so that three substances contained in the sample are detected.

### Other Embodiments

Embodiments 1 through 4 may be combined in an appropriate manner. The number of the openings independently provided in the spacer substrate 8 is not limited to two or three, and may be four or greater. The sample is not limited to blood or a liquid obtained as a result of blood being preprocessed. The first substance and the second substance are not limited to HblAc and Hb. Thus, a biosensor detecting any of various substances is provided.

A biosensor of this disclosure is preferably usable in the medical and clinical inspection, pharmaceutical manufacturing, or various other industrial fields.

While the present subject matter has been described with respect to preferred embodiments thereof, it will be apparent to those skilled in the art that the disclosed subject matter may be modified in numerous ways and may assume many embodiments other than those specifically described above. Accordingly, it is intended by the appended claims to cover all modifications of the present subject matter that fall within the true spirit and scope of the present subject matter.

What is claimed is:

1. A biosensor, comprising:
a base substrate having a main surface;
an electrode layer disposed on the main surface of the base substrate and including a first electrode pair and a second electrode pair, each pair of the first electrode pair and the second electrode pair including a working electrode and a counter electrode;
a spacer substrate disposed on the electrode layer and having a first opening and a second opening formed therein, the first opening exposing at least a part of the first electrode pair and the second opening exposing at least a part of the second electrode pair, the second opening being independent from the first opening;
a first reagent layer containing a first reagent reactive with a sample, the first reagent layer being disposed in the first opening;
a second reagent layer containing a second reagent reactive with the sample, the second reagent layer being disposed in the second opening; and
a top cover substrate disposed on the spacer substrate, the top cover substrate including an introduction portion having at least one introduction opening formed therein, the introduction portion overlapping a part of the first opening in the spacer substrate and a part of the second opening in the spacer substrate,
wherein in the spacer substrate, an area size of the first opening is larger than an area size of the second opening.

2. The biosensor according to claim 1, wherein in the electrode layer, a length of a border between the working electrode and the counter electrode adjacent thereto of the first electrode pair exposed to the first opening is longer than a length of a border between the working electrode and the counter electrode adjacent thereto of the second electrode pair exposed to the second opening.

3. The biosensor according to of claim 1, further comprising a first discharge opening and a second discharge opening in communication with the first opening and the second opening in the spacer substrate, respectively.

4. The biosensor according to claim 3, wherein the first discharge opening and the second discharge opening are provided in the spacer substrate, each have an opening at a side surface of the spacer substrate, and are connected with the first opening and the second opening, respectively.

5. The biosensor according to claim 3, wherein the first discharge opening and the second discharge opening are provided in the top cover substrate and overlap another part of the first opening in the spacer substrate and another part of the second opening in the spacer substrate, respectively.

6. The biosensor according to claim 1, wherein:
the introduction portion of the top cover substrate comprises one introduction opening formed therein, and
the introduction opening overlaps the part of the first opening in the spacer substrate and the part of the second opening in the spacer substrate.

7. The biosensor according to claim 1, wherein:
the introduction portion of the top cover substrate comprises two introduction openings formed therein, and
one of the introduction openings overlaps the part of the first opening in the spacer substrate, and another of the introduction openings overlaps the part of the second opening in the spacer substrate.

8. The biosensor according to claim 1, wherein:
the base substrate, the spacer substrate and the top cover substrate each have a strip shape that had long sides extending in a first direction longer than short sides extending in a second direction perpendicular to the first direction,
the first opening and the second opening in the spacer substrate are disposed adjacent to each other in the first direction, and the introduction portion of the top cover substrate comprises one introduction opening faulted therein, and the introduction opening overlaps parts of the first opening and the second opening that are adjacent to each other.

9. The biosensor according to claim 8, wherein:
the top cover substrate further comprises a first discharge opening and a second discharge opening formed therein,
the first discharge opening and the introduction opening in the introduction portion respectively overlap two ends in the first direction of the first opening in the spacer substrate, and
the second discharge opening and the introduction opening in the introduction portion respectively overlap two ends in the first direction of the second opening in the spacer substrate.

10. The biosensor according to a claim 1, wherein:
the sample contains hemoglobin separated from erythrocytes,
the hemoglobin contains hemoglobin Alc,
the first reagent is specifically reactive with the hemoglobin Alc or a substance derived from the hemoglobin Alc, and
the second reagent is reactive with the hemoglobin.
